(19) United States
(12) Patent Application Publication
(10) Pub. No.: US 2003/0236291 A1
(43) Pub. Date: Dec. 25, 2003

(54) Use of citreamicins

(76) Inventors: Dolores Garcia Gravalos, Tres Cantos (ES); Jose Luis Fernandez Puentes, Leon (ES); Librada Maria Canedo Fernandez, Leon (ES); Julia Perez-Baz, Leon (ES); Francisco Romero-Millan, Leon (ES); Fernando Espliego-Vazquez, Leon (ES)

Correspondence Address: Fish & Richardson, 225 Franklin Street, Boston, MA, 02110-2804, US

(21) Appl. No.: 10/276,343

(22) PCT Filed: May 17, 2001
(86) PCT No.: PCT/GB01/02148

(30) Foreign Application Priority Data
May 17, 2000 (GB) 0011927.1

(51) Int. Cl. A61K 31/42; A61K 31/352; A61K 31/122

(52) U.S. Cl. 514/378; 514/456; 514/682

(57) ABSTRACT

Citreamicins possess useful antitumor activity. Typically they are of formula, wherein R₁ is selected from the group consisting of COCH₂CH(CH₃)₂, COCH₃ or H when R₂ is CH₃, or wherein R₁ is COCH₂CH(CH₃)₂, and R₂ is H.

The present invention relates to citreamicins, and in particular to a new use of the citreamicins.

### BACKGROUND

The citreamicins are known comopunds, see: Pearce, C. J.; Carter, G. T.; Nietsche, J. A.; Borders, D. B.; Greenstein, M. and Maiese, W. M. J. Antibiotics, 1991, 44(11), 1247-1250. The citreamicins thus include compounds of the formula:

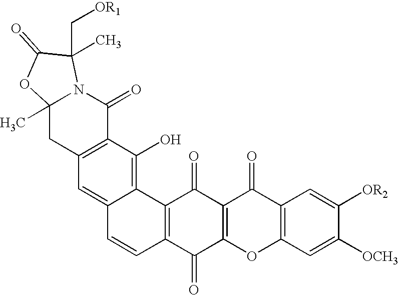

wherein R₁ is selected from the group consisting of COCH₂CH(CH₃)₂, COCH(CH₃)₂, COCH₃ or H when R₂ is CH₃, or wherein R₁ is COCH₂CH(CH₃)₂ and R₂ is H. In particular, citreamicin a is a compound of formula (I) where R₁ is COCH₂CH(CH₃)₂ and R₂ is CH₃.

### SUMMARY OF INVENTION

We have now found a new use of the known citreamicins, especially those of the formula (I). We have found that they exhibit antitumor activity.

Thus, we provide pharmaceutical compositions for treatment of tumors and which include a citreamicin and a pharmaceutically acceptable carrier.

We further provide methods of making such pharmaceutical compositions, including the use of a citreamicin in the preparation of a medicament for use in treating a tumor.

Additionally, we provide a method for treating a mammal affected by a malignant tumor sensitive to a citreamicin compound such as a compound of formula (I), which comprises administering to the affected individual a therapeutically effective amount of the citreamicin compound or a pharmaceutical composition thereof

### DETAILS OF THE INVENTION

Examples of pharmaceutical compositions include any solid (tablets, pills, capsules, granules, etc.) or liquid (solutions, suspensions or emulsions) with suitable formulation of oral, topical or parenteral administration, and they may contain the pure compound or in combination with any carrier or other pharmacologically active compounds. These compositions may need to be sterile when administered parenterally.

The correct dosage of a pharmaceutical composition comprising a citreamaicin compound will vary according to the pharmaceutical formulation, the mode of application, and the particular situs, host and tumor being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

We have found in particular that citreamicin α exhibits in vitro antitumor activity against a cell line derived from mouse lymphoma.

### BIOLOGICAL ACTIVITY

Citreamicin a displays good antitumor activity. Its antitumor activity has been detected in vitro by culturing the tumor cells following the methodology described by

(1): Raymond I. Bergeron, Paul F. Cavanaugh, Jr., Steven J. Mine, Robert G. Hughes, Jr., Gary T. Elliot and Carl W. Porter. Antineoplastic and antiherpetic activity of spermidine catecholamide iron chelators. Biochem. Bioph. Res. Comm. 1984, 121(3): 848-854; and

(2). Alan C. Schroeder, Robert G. Hughes, Jr. and Alexander Bloch. Effects of Acycic Pyrimidine Nucleoside Analoges. J. Med. Chem. 1981, 24:1078-1083.

Cells were maintained in logarithmic phase of growth in Eagle's Minimum Essential Medium, with Earle's Balanced Salts, with 2.0 mM L-glutamine, with non-essential amino acids, without sodium bicarbonate (EMEM/neaa); supplemented with 10% Fetal Calf Serum (FCS), 10⁻² M sodium bicarbonate and 0.1 g/l penicillin-G +streptomycin sulfate.

A screening procedure has been carried out to determine and compare the antitumor activity of citreamicin CL, using an adapted form of the method described by Bergeron et al. The antitumor cells employed were P388 (ATCC CCL-46, suspension culture of a lymphoid neoplasm from DBA/2 mouse), A549 (ATCC CCL-185, monolayer culture of a human lung carcinoma) and HT-29 (ATCC HTB-38, monolayer culture of a human colon carcinoma).

P388 cells were seeded into 16 mm wells at 1×10⁴ cells per well in 1 ml aliquots of MEM 5FCS containing the indicated concentration of drug. A separate set of cultures without drug was seeded as control growth to ensure that cells remained in exponential phase of growth. All determinations were carried out in duplicate. After three days of incubation at 37° C., 10% CO₂ in a 98% humid atmosphere, an approximate IC₅₀ was determined by comparing the growth in wells with drug to the growth in wells control.

A549 and HT-29 cells were seeded into 16 mm wells at 2×10⁴ cells per well in 1 ml aliquots of MEM 1OFCS containing the indicated concentration of drug. A separate set of cultures without drug was seeded as control growth to ensure that cells remained in exponential phase of growth. All determinations were carried out in duplicate. After three days of incubation at 37° C., 10% CO₂ in a 98% humid atmosphere, the wells were stained with 0.1% Crystal Violet. An approximate IC₅₀ was determined by comparing the growth in wells with drug to the growth in wells control.

The activity results, IC₅₀ (μM), for citreamicin a are given in the following table:

| | P388 | A549 | HT-29 |
|---|---|---|---|
| | ATCC CCL-46 | ATCC CCL-185 | ATCC HTB-38 |
| Citreamicin α | 0.003 | 0.004 | 0.004 |

1. The use of a citreamicin in the preparation of a medicament for the treatment of a tumor.

2. The use according to claim 1, wherein the citreamicin is of the formula (I), 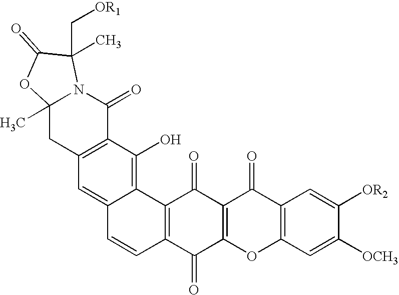
wherein R₁ is selected from the group consisting of COCH₂CH(CH₃)₂, COCH(CH₃)₂, COCH₃ or H when R₂ is CH₃, or wherein R₁ is COCH₂CH(CH₃)₂ and R₂ is H.

3. The use according to claim 2, wherein the citreamicin is citreamicin α which is of formula (I) where R₁ is COCH₂CH(CH₃)₂ and R₂is CH₃.

4. A method of treating a tumor which comprises administration of an effective amount of a citreamicin compound.

5. A method according to claim 4, wherein the citreamicin is of the formula (I), 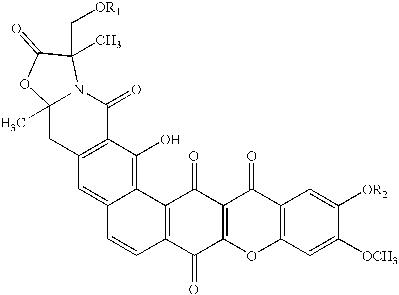
wherein R₁ is selected from the group consisting of COCH₂CH(CH₃)₂, COCH(CH₃)₂, COCH₃ or H when R₂ is CH₃, or wherein R₁ is COCH₂CH(CH₃)₂ and R₂ is H.

6. A method according to claim 5, wherein the citreamicin is citreamicin a which is of formula (I) where R₁ is COCH₂CH(CH₃)₂ and R₂ is CH₃.

7. A pharmaceutical composition with antitumor activty comprising a citreamicin and a pharmaceutically acceptable carrier.

8. A pharmaceutical composition according to claim 7, wherein the citreamicin is of the formula (I), 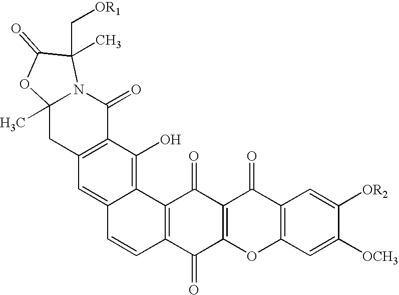
wherein R₁ is selected from the group consisting of COCH₂CH(CH₃)₂, COCH(CH₃)₂, COCH₃ or H when R₂ is CH₃, or wherein R₁ is COCH₂CH(CH₃)₂ and R₂ is H.

9. A pharmaceutical composition according to claim 8, wherein the citreamicin is citreamicin α which is of formula (I) where R₁ is COCH₂CH(CH₃)₂ and R₂ is CH₃.
